(19) United States
(12) Patent Application Publication
(10) Pub. No.: US 2014/0008301 A1
(43) Pub. Date: Jan. 9, 2014

(54) THERAPEUTIC RETRIEVAL OF TARGETS IN BIOLOGICAL FLUIDS

(71) Applicant: Nanoshell Company, LLC, Layton, UT (US)

(72) Inventors: Agnes Ostafin, Layton, UT (US); Hiroshi Mizukami, Pasadena, CA (US); Michael Batenjany, Murray, UT (US)

(21) Appl. No.: 13/936,092

(22) Filed: Jul. 5, 2013

Related U.S. Application Data
(60) Provisional application No. 61/739,724, filed on Dec. 20, 2012.
(60) Provisional application No. 61/729,942, filed on Nov. 26, 2012.
(60) Provisional application No. 61/729,948, filed on Nov. 26, 2012.
(60) Provisional application No. 61/672,682, filed on Jul. 17, 2012.
(60) Provisional application No. 61/668,032, filed on Jul. 5, 2012.

(51) Int. Cl. A61M 1/36

(52) U.S. Cl. CPC A61M 1/3679
USPC 210/663; 210/200; 210/202; 252/184

(57) ABSTRACT

Method and apparatus for removing high density particles from a biological fluid such as blood using aphaeresis. The particles are preferably sub-micron in size and denser than normally occurring components of the fluid and can be removed by a modified reverse-flow gradient density centrifuge without damaging the fluid. The particles can be provided to a patient in vivo or added to the fluid after it is removed from the patient. Some particles can carry and deliver oxygen and scavenge carbon dioxide. Other particles are conjugated to capture molecules for attaching to targets such as cancer cells, viruses, pathogens, toxins, or excess concentrations of a drug or element in the fluid. The targets are then removed from the fluid along with the particles by the aphaeresis instrument.

### CROSS-REFERENCE TO RELATED APPLICATIONS

This application claims priority to and the benefit of the filing of U.S. Provisional Patent Application Ser. No. 61/739,724, entitled “Therapeutic Reverse-Flow Density Gradient (RFDG) Aphaeresis”, filed on Dec. 20, 2012; U.S. Provisional Patent Application Ser. No. 61/729,942, entitled “Retrieval of Iron and Other Divalent Metals in the Plasma with Reverse-Flow Density Gradient (RFDG) Centrifugation”, filed on Nov. 26, 2012; U.S. Provisional Patent Application Ser. No. 61/729,948, entitled “Retrieval of Chemotherapeutic Agents and Metastatic Cancer Cells from Blood with Reverse-Flow Density Gradient (RFDG) Centrifugation”, filed on Nov. 26, 2012; U.S. Provisional Patent Application Ser. No. 61/672,682, entitled “Retrieval Viruses in the Plasma with Reverse-Flow Density Gradient (RFDG) Centrifugation”, filed on Jul. 14, 2012; and U.S. Provisional Patent Application Ser. No. 61/668,032, entitled “Retrieval of High-Density Particle Conjugated Hemoglobin in the Plasma with Reverse-Flow Density Gradient (RFDG) Centrifugation”, filed on Jul. 5, 2012. This application is also related to U.S. patent application Ser. No. 13/322,757, entitled “Synthesis of Oxygen Carrying, Turbulence Resistant, High Density Submicron Particulates”, which claims priority to PCT application Serial No. PCT/US10/46417, filed on Aug. 24, 2010, and U.S. patent application Ser. No. 13/322,790, entitled “Method and Apparatus for Continuous Removal of Submicron Sized Particles in a Closed Loop Liquid Flow System”, which claims priority to PCT application Serial No. PCT/US2010/046421, filed on Aug. 24, 2010, both of which PCT applications claimed priority to and the benefit of the filing of U.S. Provisional Patent Application Ser. No. 61/236,810, filed on Aug. 25, 2009. The specifications and claims of all of these applications are incorporated herein by reference.

### FEDERALLY SPONSORED RESEARCH OR DEVELOPMENT

The U.S. Government has a paid-up license in this invention and the right in limited circumstances to require the patent owner to license others on reasonable terms as provided for by the terms of Contract Nos. HHSN268201200059C and 3R41HL095250-01A1S1 awarded by the U.S. National Institutes of Health.

### BACKGROUND OF THE INVENTION

1. Field of the Invention (Technical Field)

Embodiments of the present invention are related to retrievable nanoparticles that can be mixed with a patient's blood and that are capable of selectively binding to target molecules, ions, viruses and/or cells for removal from the blood stream of a patient. Other embodiments of the present invention are related to a low-cost, continuous reverse-flow density gradient centrifuge (RFDGC) that can perform this retrieval, either extracorporeally or corporeally, optionally comprising an efficient continuous mixing device for mixing the retrievable nanoparticles and the target pathogens in the patient's blood without damaging other blood components. It is thus possible with embodiments of the present invention to therapeutically treat patients while minimizing side effects resulting from, for example, metabolized drugs, overdosed or unused drugs and particles such as imaging particles are removed from the bloodstream before they can become the secondary cause of toxins.

2. Background Art

Note that the following discussion refers to a number of publications and references. Discussion of such publications herein is given for more complete background of the scientific principles and is not to be construed as an admission that such publications are prior art for patentability determination purposes.

Many adverse health conditions result in the accumulation of abnormal compositions in the blood. For example, excess drugs given to patients, self consumed drugs and alcohol, and other consumables may be reflected in abnormal blood compositions. Some of the components found in the blood of affected individuals are used as diagnostic markers for disease and health conditions, while others may contribute to further problems by causing secondary symptoms and conditions. Medical treatment in such cases aims at reducing, masking or counteracting the effects of specific molecules and toxins, and supporting body processes that facilitate their clearance from the body. When symptoms become severe, the body can no longer handle the abnormal compositions, and facilitated medical intervention may be insufficient. Patients may suffer permanent damage or even death as a result.

Aphaeresis is a well-established clinical method that is used to separate components of blood for treatment or donation. In this method components of the blood which span a relatively narrow range of densities, but a much wider range of molecular weight and size, can be efficiently, rapidly, and continuously separated. Because blood is a heterogeneous non-ideal fluid, and most of the molecules and/or cells that are diagnostic for a disease fall within the density range spanned by the largest and smallest blood components, conventional aphaeresis is often ineffective as an exclusive therapeutic device to collect and reduce the body's disease load, except possibly in cases where a major blood component is exchanged for a similar component obtained from healthy individuals (e.g. transfusion of red blood cells, harvesting of stem cells, platelets, white cells, removal of defective cells, etc.). Small molecules and ions are inseparable from plasma, and larger macromolecules and their complexes may co-separate with a vital blood component that should not be removed. Even some cells like metastatic cancer cells and stem cells are similar enough to normally present blood corpuscles to make separation difficult.

There are many situations where separation of targets from non-ideal fluids is desirable. For example, patients with malignant cancer are typically treated with radiation and chemotherapeutic agents by scheduled infusion. Despite these efforts for many cancers the frequency of recurrence and metastasis remains significant. In some cases the cancer may be kept at bay if evidence for recurrence or continued malignancy could be caught early and the therapy modified appropriately. However, detecting evidence for metastatic cancer cells at early stages is difficult. By the time imaging and blood chemistry markers are able to reveal a problem the recurrence is well underway and more aggressive treatment is required. Exacerbating this problem is the potential for some cancer cells to change their resistance to drug over time, which suggests that if effective detection and eradication could occur at early stages then patient prognosis could be significantly improved. Moreover, when present in sufficient quantity, certain cancer cells can be isolated from tissues such as blood and detected using immunological methods. Other types of cancer cells lack unique receptors and cannot be isolated in this manner, but they can be located via conventional staining procedures by laboratory technicians. Diagnosis therefore relies on the ability to find these diseased cells, which may be present in exceedingly small quantities.

In another example, treatments of some viral diseases are available and preventable by immunization, but others are not. A patient who has not been immunized for a viral disease may be able to develop natural immunity and eventually build resistance to the disease. During this period, however, the patient may suffer from fever, infection and even life threatening symptoms, despite the intervention of indirect treatments to ease symptoms. Thus, being able to directly reduce the proliferation of viruses in the body of patients in crisis situations could bring significant benefits leading to recovery from viral diseases. In addition, it has become evident that methods to remove viruses (e.g., HIV, Ebola, or Hepatitis C) early in the infection greatly impacts prognosis and the effect of therapeutic treatments. Similarly, studies have shown that reducing the initial exposure load to toxins (e.g. snake bites, bacterial, or insect bites) can dramatically affect recovery, even avert death. Whether or not anti-toxin is available, by reducing the initial toxin load by removing toxins from the blood, neurotoxic, hemotoxic, necrotic, and other damage, as well as time spent in the hospital, may be minimized and disfigurement and death prevented.

In another example, patients with chronic hemoglobinopathies and other hemolytic diseases are typically treated by regular transfusion to replace lost oxygen carrying function and remove defective cells and their breakdown products. However, unlike the blood cells normally produced in the body, transfused blood cells are more fragile and tend to break down quicker in the blood stream. This leads to release of free iron from hemoglobin into the blood and eventual accumulation of iron in tissues and organs since the normal transferrin/ferritin network becomes overloaded and clearance of iron from the body cannot keep up. In those individual with defective iron clearance systems such as those with iron overload syndrome, the problem is even more acute. Chelation therapeutics are drugs that are taken by patients exhibiting signs of excessively high iron levels. Taken by injection or orally these drugs supplement the transfusion therapy and prolong patient well-being and avoid crisis. Compliance with injection regimes has been difficult, but greatly improved with new oral medications. Bioavailability of oral drugs is still problematic and side effects arising from the larger than needed dosages that must be taken to reach consistent therapeutic levels is still an issue. The ability to lower iron levels in the blood could reduce side effects of transfusion therapy as well as alleviate the suffering of those with excess iron in the blood. In case of sickle cell anemia, the red blood cells (RBC) of patients are hemolyzed and their hemoglobin (HbS) released in the plasma becoming a cause of severe oxidative stress. Specifically, intravascular release of the tetrameric Hb results in its disassociation into a dimeric form. As a reactive molecule, hemoglobin can generate oxidant species. Outside a red blood cell, hemoglobin can react with plasma compounds, leading to oxidations. Free hemoglobin is linked to the susceptibility of deoxyhemoglobin to oxidation, leading to the production of methemoglobin, which has a peroxidative activity and forms further reactive O₂ species. Oxidation of methemoglobin also releases hemin, which rapidly associates with membranes, leading to cytotoxicity. The Hb scavenger Haptoglobin (Hp) will irreversibly bind the dimeric form of Hb. The Hp-Hb complex can associate with the receptor CD163, found on the surface of monocytes and macrophages and then endocytosed for removal by degradation. However, when the binding capacity of plasma proteins is overwhelmed, the hemoglobin can reach and overload the absorptive capacity of the kidney (hemoglobinuria), leading to nephrotoxicity.

As mentioned, treatment of these diseases mostly relies on drugs, high energy radiation, temperature, immunity, etc., which usually take place while these pathogens still reside in the body of the patient, which causes unwanted side effects. Thus there is a need for a therapeutic method that removes those pathogens from the blood circulation of the patient.

### SUMMARY OF THE INVENTION (DISCLOSURE OF THE INVENTION)

An embodiment of the present invention is a method for introducing and removing high density particles from a biological fluid, the method comprising introducing high density particles into a biological fluid, the high density particles comprising a density greater than any naturally occurring component of the biological fluid, and removing at least some of the high density particles from the biological fluid using aphaeresis. The introducing step can be performed in vivo or extracorporeally, in which case the method preferably comprises transferring the biological fluid from a patient prior to the introducing step. The particles optionally deliver oxygen in the biological fluid and scavenge carbon dioxide from the biological fluid. The method preferably further comprises conjugating the particles to one or more capture molecules, and preferably further comprises attaching at least some of the particles to one or more targets in the biological fluid via the capture molecules prior to the removing step. The removing step preferably comprises removing at least some of the attached targets from the biological fluid. The targets can optionally attach to an intermediary which is attached to the capture molecules. The intermediary optionally comprises haptoglobin in which case the targets comprise hemoglobin. The method preferably further comprises minimizing opsonization, adherence to cells, and interaction of the particles with non-target components of the biological fluid or organs in a patient by adjusting the size and/or surface properties of the particles. The method preferably further comprises incorporating PEGylated and/or neutral lipids at the surface of the particles.

The removing step is preferably performed using a reverse-flow density gradient (RFDG) centrifuge. The method preferably further comprises mixing the particles with the biological fluid in a mixing chamber, the mixing chamber comprising one or more spiral tubes. The flow of the particles and the biological fluid is substantially lamellar within each spiral tube but not between spiral tubes. The method preferably further comprises automatically adjusting a removal efficiency of the particles by monitoring the concentration of particles in the biological fluid. Each of the high density particles preferably comprises a composition selected from the group consisting of a core comprising perfluorocarbon surrounded by a surfactant, a surface modified solid core; and an activated magnetic bead. The surfactant preferably comprises a phospholipid-based monolayer. The solid core preferably comprises one or more nanoparticles comprising gold, silver, titanium, iron, silica, or a ceramic. The method preferably further comprises returning the biological fluid to a patient after the removing step. The biological fluid is preferably blood.

Another embodiment of the present invention is a particle capable of being removed from a biological fluid by aphaeresis, the particle comprising either a core comprising perfluorocarbon surrounded by a surfactant layer or a surface modified solid core, the particle comprising a density greater than any naturally occurring component of the biological fluid. The perfluorocarbon preferably comprises perfluoroctanylbromide. The surfactant layer preferably comprises a phospholipid-based monolayer. The phospholipid preferably comprises a neutral or negatively charged headgroup. The phospholipid preferably comprises a monounsaturated and/or neutral phospholipid. The phospholipid optionally comprises 18:1 DOPC or 18:1 DOPA. The layer preferably comprises a co-surfactant comprising a functionalized headgroup for conjugating a capture molecule and/or a PEGylated phospholipid. The PEGylated phospholipid preferably comprises between 10 and 40 PEG subunits, and more preferably between 14 and 25 PEG subunits. The PEGylated phospholipid optionally comprises PEG1000PE. The co-surfactant optionally comprises 18:1 Dodecanyl PE or 1,2-dioleoyl-sn-glycero-3-phosphoethanolamine-N-(dodecanyl). The particle optionally delivers oxygen in the biological fluid and/or scavenges carbon dioxide from the biological fluid. The solid core preferably comprises one or more nanoparticles comprising gold, silver, titanium, iron, silica, or a ceramic and is preferably surface modified with Thiol-PEG-COOH or HO-PEG-NH₂ groups.

Another embodiment of the present invention is an apparatus for removing high density particles from a biological fluid extracorporeally, the apparatus comprising a mixing chamber for mixing the high density particles with the biological fluid and a reverse-flow density gradient (RFDG) centrifuge, wherein a density of the high density particles is greater than any naturally occurring component of the biological fluid. The mixing chamber preferably comprises a spiral tube, and optionally comprises a plurality of spiral tubes connected in series. The flow of the particles and the biological fluid is preferably substantially lamellar within each spiral tube but not in a region connecting two spiral tubes. The apparatus preferably further comprises a pump for pumping the biological fluid through the mixing chamber and a syringe pump located before an inlet to the mixing chamber for combining the high density particles with the biological fluid. The centrifuge preferably comprises a variable element, the element selected from the group consisting of spin rate, number of open outlet ports, and flow rate of liquid through each outlet port.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated into and form a part of the specification, illustrate an embodiment of the present invention and, together with the description, ‘serve to explain the principles of the invention. The drawings are only for the purpose of illustrating various embodiments of the invention and are not to be construed as limiting the invention. In the drawings:

FIG. 1 is a schematic of an embodiment of a high-density particle of the present invention.

FIG. 2 is a schematic of an embodiment of a high-density particle of the present invention conjugated to a target specific ligand.

FIG. 3 is a schematic drawing of targets bound to a high-density submicron particle through attached capture molecule. For example, the targets may be viruses, and the capture molecules may comprise antibodies. Or, the targets may be iron compounds or particles (in various oxidation states) and the capture molecules may comprise chelators. A mixture of different particles may be made into a cocktail to retrieve multiple different targets simultaneously.

FIG. 4 is a schematic diagram showing corporeal retrieval of targets conjugated with high-density submicron particles in the blood with the reverse flow density gradient (RFDG) aphaeresis system in accordance with embodiments of the present invention.

FIG. 5 is a schematic diagram showing extra-corporeal retrieval of high-density submicron particles in the blood in accordance with embodiments of the present invention. In this arrangement, the high density submicron particles do not enter the patient's body.

FIG. 6A is a front view of an embodiment of a spiral mixing element of the present invention.

FIG. 6B is a side view of an embodiment of a mixing chamber of the present invention comprising multiple stacked mixing elements.

FIG. 7 is a schematic drawing of haptoglobin binding to a high-density submicron particle through an antibody to haptoglobin, thereby forming the complex rHDP-Hp.

FIG. 8 is a schematic drawing of indirect binding of what could be more than 700 sickle cell hemoglobin (HbS) molecules to rHDP-Hp through haptoglobin and its antibody, thereby forming the complex rHDP-Hp-Hb.

FIG. 9 is a graph showing results of Example 3 demonstrating magnetic bead (MB) separation in phosphate buffered saline (PBS) with a Cobe Spectra aphaeresis instrument. Clear separation of MB is noted at 2,400 rpm.

FIG. 10 shows graphs of results from Example 5 comparing iron chelation using free Desferoxamine (DFO) and DFO-MB.

### DETAILED DESCRIPTION OF THE INVENTION

As used throughout the specification and claims, the terms “capture molecule” or “target specific ligand” or “TSL” mean any moiety that selectively binds to both a target and embodiments of submicron particles of the present invention, including but not limited to ion, metal, chelator, lectin, haptoglobin, aptamer, DNA, nucleic acid fragment or sequence, ligand, antigen, antibody, protein nucleic acid, enzyme, macrophage, chemotherapy reagent, and the like. A capture molecule may also be any natural, synthetic or recombinant protein, fragment, sequence or molecule which, when attached to a high-density submicron particle retains its ability to form a stable complex with a desired target. As used throughout the specification and claims, the term “target” means a specific molecule, drug, cell fragment, cell, pathogen, toxin, poison, DNA, nucleic acid, nucleic acid fragment or sequence, peptide, antibody, antibody fragment, protein, polysaccharide, divalent metal, virus, fungus, bacterium, mycoplasm, and the like, typically associated with a disease or combination of diseases, or the equivalent thereof.

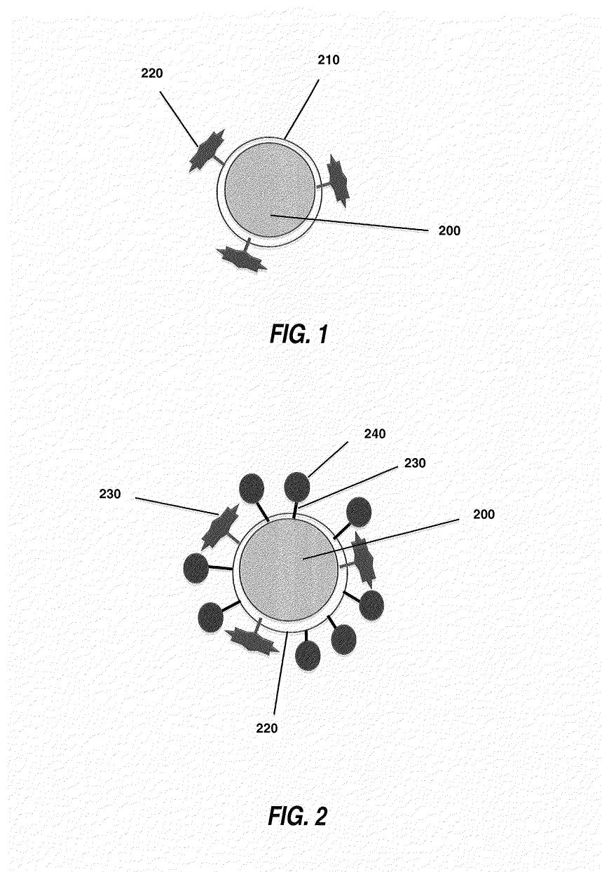

Embodiments of the present invention comprise high-density particles that can be delivered into a biological fluid either in vivo or extra-corporeally, and which are retrievable by aphaeresis methods. One embodiment, shown in FIG. 1, comprises a PFC-containing emulsion, which comprises perfluorocarbon 200, for example perfluoroctanylbromide (PFOB), core surrounded by surfactant 210. The surfactant preferably comprises a phospholipid-based monolayer. The major surfactant in the monolayer typically comprises a monounsaturated neutral phospholipid, such as 18:1 (Δ9-Cis) PC (DOPC), which is 1,2-dioleoyl-sn-glycero-3-phosphocholine, but may alternatively comprise a monounsaturated negative headgroup phospholipid such as 18:1 (Δ9-Cis) PA (DOPA) which is 1,2-dioleoyl-sn-glycero-3-phosphate. The monolayer preferably also comprises PEGylated phospholipid 220 to stabilize the structure, prevent aggregation, and offer stealth by preventing opsonization and adherence to cells, thereby increasing circulation half-life. The PEGylated phospholipid typically comprises approximately 10-40 PEG subunits and a PEGylated 18:1 DOPE derivative, for example 18:1 PEG1000 PE which is 1,2-dioleoyl-sn-glycero-3-phosphoethanolamine-N-[methoxy(polyethylene glycol)-1000], in which case the PEG portion comprises 22 subunits.

In some embodiments where attaching the high density particle to a target is desired, the monolayer will also preferably comprise a DOPE derivative 230 with an extended headgroup terminating in a carboxyl or amine group used to conjugate the desired target specific ligand (TSL), as shown in FIG. 2. In some embodiments the derivative comprises an 18:1 Dodecanyl PE which is 1,2-dioleoyl-sn-glycero-3-phosphoethanolamine-N-(dodecanyl) (DD-DOPE). The headgroup on DD-DOPE has an extension of approximately 2.3 nm ending in a carboxyl group. An antibody or other desired TSL 240 can be covalently attached, for example using 1-Ethyl-3-[3-dimethylaminopropyl]carbodiimide hydrochloride (EDC) and Sulfo-(N-hydroxysulfosuccinimide) (S-NHS) chemistry. The final product typically has a diameter between 200-300 nm, which can be determined by, for example, dynamic light scattering (DLS). The PFC core typically is from approximately 4-20% by volume, the total surfactant is from approximately 0.2-1.5% by weight. Within this surfactant composition by molar %, for example, the major surfactant (DOPC or DOPA), the DD-DOPE, and the PEGylated phospholipid preferably range from approximately 85-90%, 8-10% and 4-5% respectively. The major volume typically comprises phosphate-buffered saline (PBS) or normal saline (0.9% NaCl).

Other embodiments of the high density particles comprise a solid core preferably comprising one or more nanoparticles such as 100 nm spheres comprising gold, titanium, silver, iron, silica, or a ceramic. The core is then surface modified, for example with ˜5000 MW Thiol-PEG-COOH or HO-PEG-NH₂ groups.

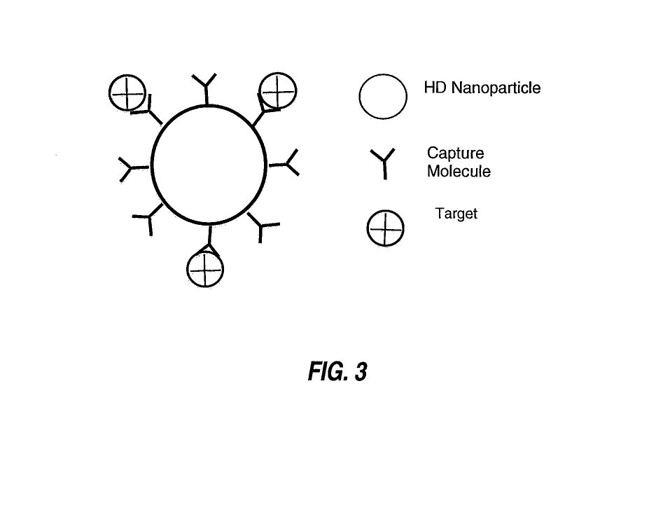

An embodiment of the invention is submicron high-density particles as described above able to bind to a specific target in the blood or in another ideal or non-ideal fluid, including biological fluids such as, but not limited to, blood, plasma, urine or cell lysates, thereby forming the complex rHDP-X, where X specifies the target or in some cases (such as haptoglobin) an intermediary that attaches to the particles and also attaches to the target. A more generalized version of this complex than FIG. 2 is shown in FIG. 3. The use of high-density particles accentuates the slight difference in density between some targets, such as cancer cells, and normal healthy cells, which facilitates retrieval of very small quantities or concentrations of targets from blood or other biological fluid, preferably using aphaeresis and reverse-flow density gradient centrifugation. The core is preferably covalently bonded to capture molecules using conventional cross-linking chemistries. Once they are in contact with a patient's blood they will bind to their intended targets, for example via binding receptors or other markers on the surface of the targets. Multiple types of rHDP-X may be used as a cocktail formulated for specific and simultaneous removal of different targets associated with a disease or diseases, such as those often afflicting immuno-compromised patients. Some examples of high density particles that are unconjugated or conjugated to form the rHDP-X complex are listed in Table 1.

TABLE 1

| | | Layer 1 | Layer 1 | | | | |
|---|---|---|---|---|---|---|---|
| | | major | minor | | Other | Primary | |
| X | core | components | components | Crosslinking | molecules | target (e.g.) | Uses |
| none | PFOB | DOPC | PEG- | EDC/S-NHS | none | Dissolved | O₂ delivery/ |
| | PFD | (DOPA) | containing | or other | | gases | CO₂ |
| | | | lipid | | | | scavenger |
| anti- | PFOB | DOPC | PEG- | EDC/S-NHS | Anti- | haptoglobin | research |
| haptoglobin | PFD | (DOPA) | containing | or other | haptoglobin | | |
| | | | lipid | | | | |
| haptoglobin | Same | Same | Same | Same | haptoglobin | hemoglobin | SCD |
| Chelator | Same | Same | Same | Same | desferox- | Iron | Therapeutic |
| | | | | | amine or | | scavenger |
| | | | | | similar | | |
| lectin | Same | Same | Same | Same | Lectin | Fungus, | Therapeutic |
| | | | | | | toxins, | scavenger |
| | | | | | | carbohydrate | |
| other | Same | Same | Same | Same | Various | Virus, | Therapeutic |
| | | | | | | chemo- | scavenger |
| | | | | | | theraputics, | |
| | | | | | | cells, toxins | |

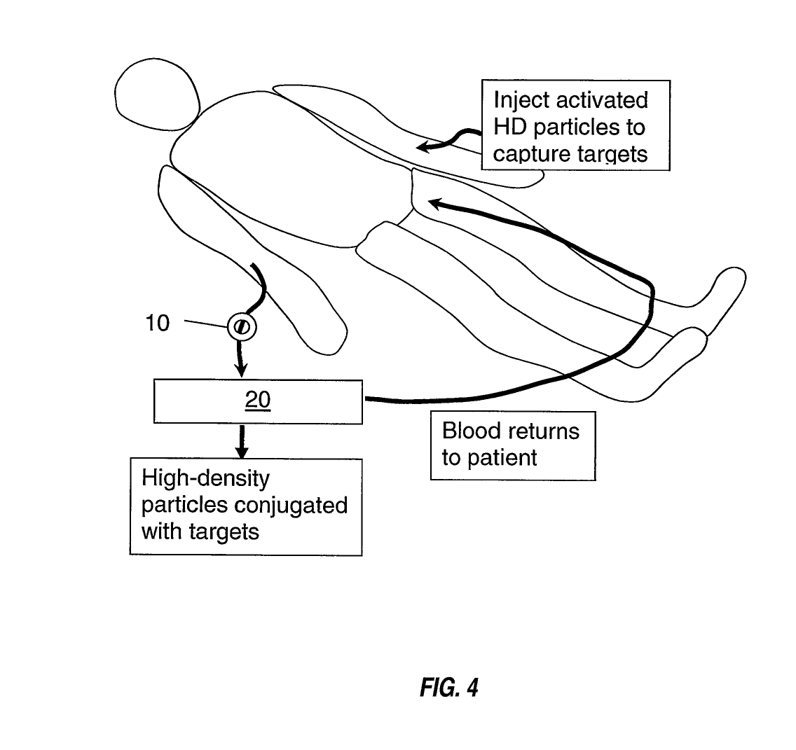

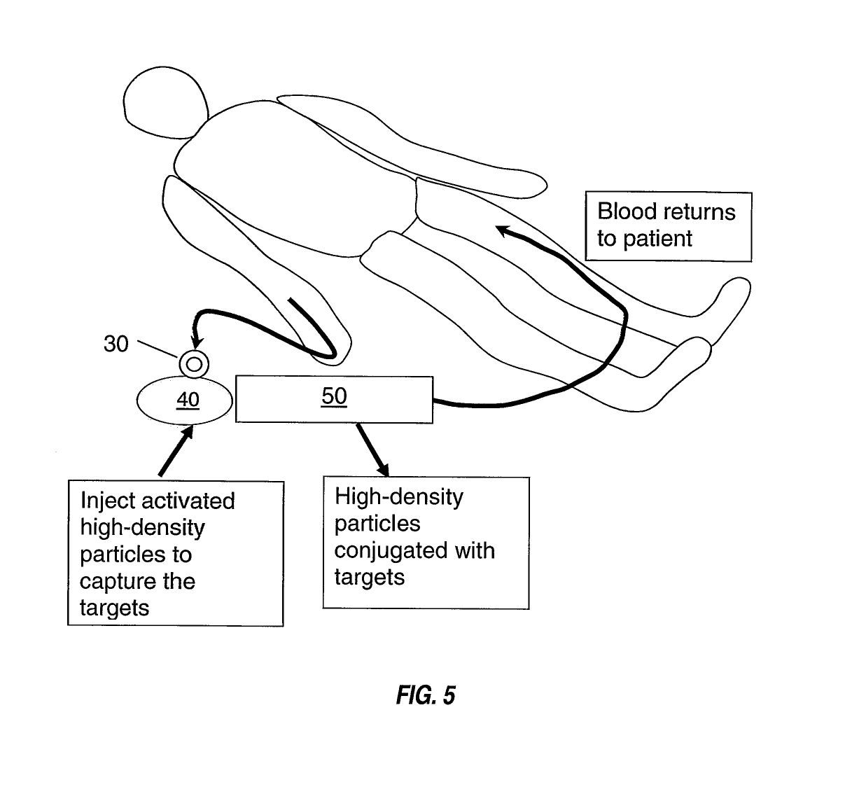

The rHDP-X is retrieved or removed from the patient's blood, preferably using an aphaeresis system comprising reverse-flow density gradient (RFDG) aphaeresis cell-sorter, such as that disclosed in U.S. patent application Ser. No. 13/322,790. Some embodiments of the system may be portable and battery and/or solar powered, enabling use in locations where there is minimal technological infrastructure. In one embodiment, shown in FIG. 4, the particles are introduced into the patient's blood, such as via intravenous injection, for example for therapeutic purposes such as a chemotherapy infusion or to capture targets. The blood/particle mixture is pumped via pump 10 to reverse flow density gradient cell sorter 20, which separates out the particles and returns blood to the patient without the particles or targets, and preferably without damage to the blood or healthy blood cells. In another embodiment, shown in FIG. 5, the patient's blood is pumped via pump 30 into mixing chamber 40, where particles are mixed with the patient's blood extra-corporeally, preferably at the inlet of the aphaeresis instrument (reverse flow density gradient cell sorter 50), which removes the conjugated particles and returns the blood to the patient. In this embodiment the particles never enter the patient's body. Pump 10 or pump 30 may be located before or after RFDG cell sorter 20 or RFDG cell sorter 50, respectively.

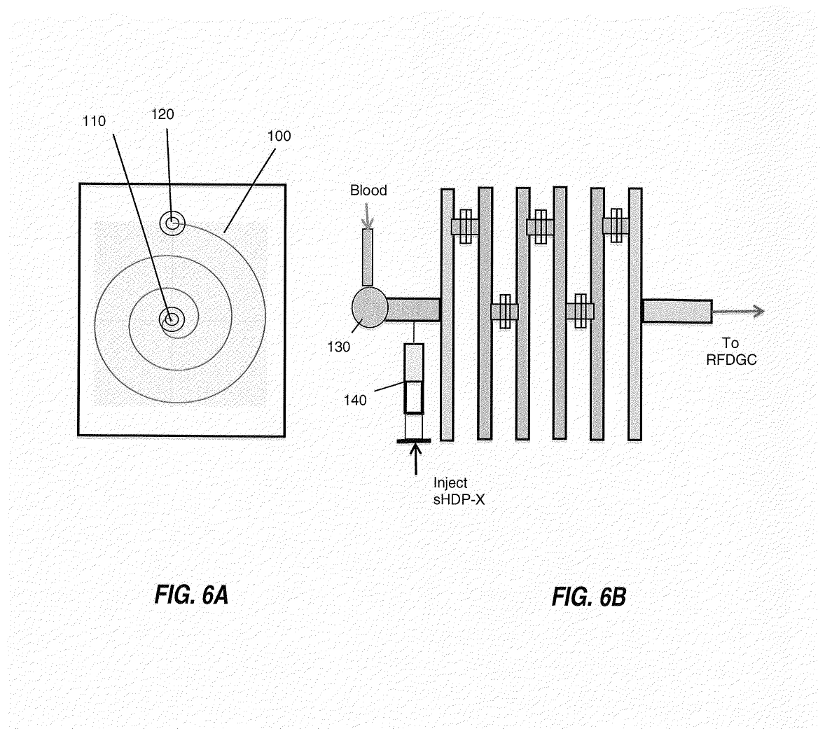

As shown in FIG. 5, extra-corporeal aphaeresis preferably utilizes a mixing chamber which facilitates the activated high-density nanoparticles binding to the targets without damaging blood components, especially the red blood cells, white cells and proteins within the range of volume and liquid flow rate acceptable to the reverse flow density gradient centrifuge (RFDGC). As shown in FIG. 6A, a mixing chamber preferably comprises an element comprising spiral tube 100 mounted on a plate, tube 100 having inlet 110 and outlet 120. Spiral tube 100 preferably comprises only curved portions (as shown) with no straight portions. Blood is preferably pumped into inlet 110 via pump 130 at an appropriate flow rate, while activated rHDP-X is injected preferably with syringe pump 140 at a controlled rate. Any number of spiral elements may be stacked in series, as shown in the side view shown in FIG. 6B, and eventually the mixture will exit the mixing chamber and enter the RFDGC. In the tubing, the particles and blood preferably undergo lamellar flow while being mixed for a desired duration of time as the reactants pass through each spiral element. Relatively smooth lamellar flow, however, will typically be interrupted in the region where the first element is connected to the second element, thereby improving mixing of the blood and the rHDP-X. This process may be continued as needed by increasing the number of elements used. The mixing efficiency may thus be varied widely by controlling the rate of blood flow, the configuration of spiral tubing (including its diameter), and the number of elements used.

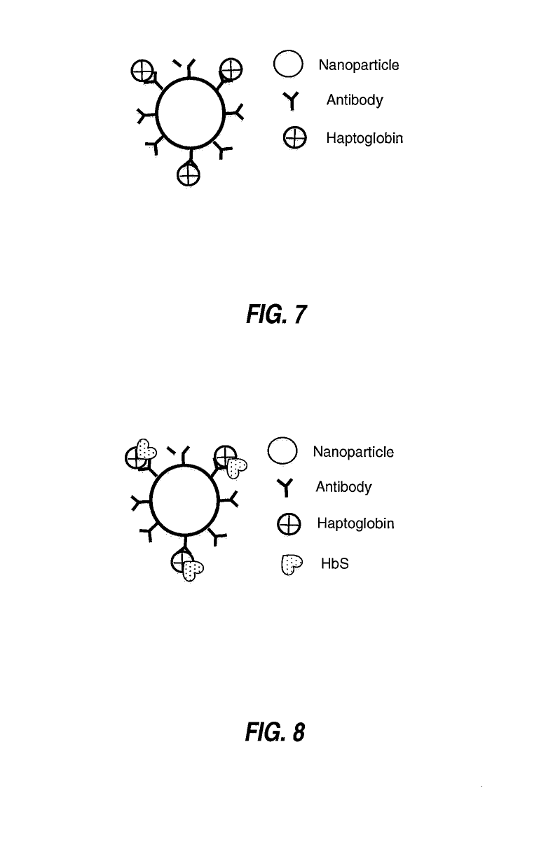

One embodiment of the invention may be used to remove hemoglobin from plasma. The level of hemoglobin in blood plasma is known to increase among patients with hemolytic anemia, sickle cell anemia, thalassemia etc. Furthermore, chronic blood transfusion to these patients could further increase the level of free hemoglobin in the blood, and they may suffer from oxidative stress. Particles of the present invention can bind a large number of hemoglobin molecules (Hb), such as hemoglobin HbA, hemoglobin HbS, etc., using capture molecules comprising antibodies of haptoglobin (Hp) or by direct conjugation of Hp to the high-density submicron particles. In this embodiment Hp is the target, and the particles form a complex with Hp (rHDP-Hp) as shown in FIG. 7. Alternatively Hb is the target for the Hp-conjugated high-density submicron particles. The complex will be able to collect Hb in the plasma, forming rHDP-Hp-Hb (FIG. 8) due to the high affinity of Hp to Hb. The Hp irreversibly binds Hb with high affinity (Kd ˜10⁻¹⁵ M) and fast rate constant (˜5.5×10⁵ M⁻¹s⁻¹). The rHDP-Hp may be injected intravenously into circulating blood, as shown in FIG. 4. The haptoglobin preferably comprises human haptoglobin, preferably Haptoglobin 1-1. This therapy will be useful to treat patients with sickle cell anemia, Thalassemia, other anemic diseases, certain bacterial infections, certain snake-bites or drugs, or those undergoing surgery or suffering from injuries.

Adjustment of the size and surface properties of the rHDP-X complexes, and/or use of PEGylated and/or neutral lipids, ensures that the particles will make minimal contact with non-target blood components, bone marrow, the liver, or any other organs, thus minimizing or preventing opsonization and adherence to cells, thereby enhancing circulation half-life and limiting toxicity. Residence or mixing time of the rHDP-X with blood can be dynamically adjusted in the aphaeresis unit to maximize capture and recovery. The retrieval efficiency preferably self-adjusts by automatically recording the remaining content of the target material in the blood or other fluid.

Other embodiments of the present invention utilize some embodiments of rHDP-X for targeting and other embodiments of rHDP-X for drug delivery to form a theranostic cocktail. For example, such a cocktail could be used for the simultaneous delivery of chemotherapeutic or photodynamic therapy agents in addition to the capture and retrieval of cancer cells from blood. The nanoparticles may also carry chemotherapeutic, photodynamic or other therapeutics, and/or radiographic or MRI imaging molecules or substances in order to perform multiple functions, including, for example, particle tracking, thus enabling improved diagnosis monitoring of the effectiveness of treatment of the disease.

The present invention can improve a patient's health status in measurable ways in cases where one or multiple disease states coexist for which removal of metabolic reaction products, defective proteins or polysaccharides and other toxic or irritating substances leads to amelioration or symptoms and lessening of the toxic load on the immune, renal or hepatic systems. In addition to removing difficult to clear metabolic by-products of drugs, embodiments of the present invention may be used to treat and diagnose or prognose various types of cancers, viral infections, fungal infections, or bacterial infections, to reduce side effects of chemotherapy, and to reduce the level of toxins, alcohol and drugs in the blood. In some embodiments patients may be treated with high drug doses while minimizing side effects resulting from metabolized drugs, since unused or residual drugs and particles are preferably removed from the bloodstream.

### EXAMPLES

### Example 1

### Oxygen Carrying Capacity of High-Density Particles

Retrievable high-density submicron particles (rNP) were formulated using 3.1 mmol 1,2-dioleoyl-sn-glycero-3-phosphocholine (DOPC), 163 μmol 1,2-dioleoyl-sn-glycero-3-phosphoethanolamine-N-[methoxy(polyethylene glycol)-1000], 20% Vol perfluoroctanylbromide (PFOB) and 80% Vol PBS. The lipids (in chloroform) were mixed, rotovaped to dryness and vacuum dessicated for 3 days. They were reconstituted in 120 ml PBS. PFOB (30 ml) was added and the mixture emulsified (5000 rpm) for 1 minute to form uniform emulsion. The emulsion was homogenized at 30K×psi×10 passes to form 230 nm particles. The formulation was split and half stored at room temperature (21° C.) and half stored at 37° C. These particles were used as oxygen carriers. A stop-flow apparatus was used to determine the oxygen capacity of the particles, although any method detecting the spectral change of deoxygenated hemoglobin upon mixing could have been used. At 20% Vol PFOB is expected to carry ˜19.9 Mol % O₂, The O₂ capacity of the formulation stored at room temperature and 37° C. was 16.7 Mol % and 14.8 Mol %, respectively. These particles are also expected to be CO₂ scavengers.

### Example 2

### Scavenging of Hb Using rNP-Hp and MB-Hp

To test Hb scavenging, normal human plasma from a blood bank was spiked with different amounts of Hb (0.4-2.0 nmol) from hemolyzed RBC to simulate the slightly hemolyzed blood of patients with sickle cell anemia (SCA). We used an accepted Hb detection assay from Arbor Assays (Ann Arbor, Mich.), which exhibits good sensitivity. We were unable to detect hemoglobin in the normal plasma obtained from a healthy donor, but detected Hb in the spiked samples as low as 1 μM. In this experiment, 250 μl of preps (Hp-rNP) using DOPC as the primary surfactant and Hp conjugated to the surface via a carboxy-terminal DOPE-derivative (DD-DOPE) with and without added PEGylation were used. Additionally, a preparation involving Hp conjugated to an activated NHS-Magnetic Bead (MB) was run alongside these preps. Hemoglobin was added to the test formulations and incubated for 30 min at room temperature by end-end mixing using a tube rotator. The rNP-Hp preps were then centrifuged at 16K×15 min to collect the supernatants. The MB-Hp preparation supernatants were collected using a magnetic stand. These supernatants were tested for free (unbound) Hb. Table 2 is a summary of the results, which suggest: (a) both rNP preparation and MB scavenge Hb; (b) the stoichiometry of Hb:Hp for the rNP preps appears to be approximately 1:2, which implies that (I) 50% of the bound Hp is conjugated at a crucial domain on Hp required for Hb capture, or (ii) 50% of the Hp is sterically hindered/blocked and inaccessible to Hb, or (iii) 50% of the Hb may be non-dimeric; c) PEGylation does not affect the ability of Hp to bind Hb.; and d) the Hp-MB complex is less efficient at scavenging Hb than the rNP-Hp formulation.

TABLE 2

| | Prep | | | | | | | | |
|---|---|---|---|---|---|---|---|---|---|
| | Prep | | | Prep | | | Prep | | |
| | without PEG9 | | | With PEG9 | | | With NHS-MB | | |
| Hp/ | 1.9 | | | 1.9 | | | 0.6 | | |
| 250 ul | | | | | | | | | |
| (nmol) | | | | | | | | | |
| Hb | 0.4 | 0.5 | 2.0 | 0.4 | 0.5 | 2.0 | 0.4 | 0.5 | 2.0 |
| added | | | | | | | | | |
| (nmol) | | | | | | | | | |
| Hb | 0 | 0 | 0.9 | 0 | 0 | 1.1 | 0 | 0 | 1.7 |
| un- | | | | | | | | | |
| bound | | | | | | | | | |
| (nmol) | | | | | | | | | |
| Hb | 0.4 | 0.5 | 1.1 | 0.4 | 0.5 | 0.9 | 0.4 | 0.5 | 0.3 |
| bound | | | | | | | | | |
| (nmol) | | | | | | | | | |
| Hb | 100 | 100 | 55 | 100 | 100 | 45 | 100 | 100 | 15 |
| bound | | | | | | | | | |
| (%) | | | | | | | | | |

### Example 3

### High-Density Particles may be Retrieved Up to 100% with an Aphaeresis Instrument

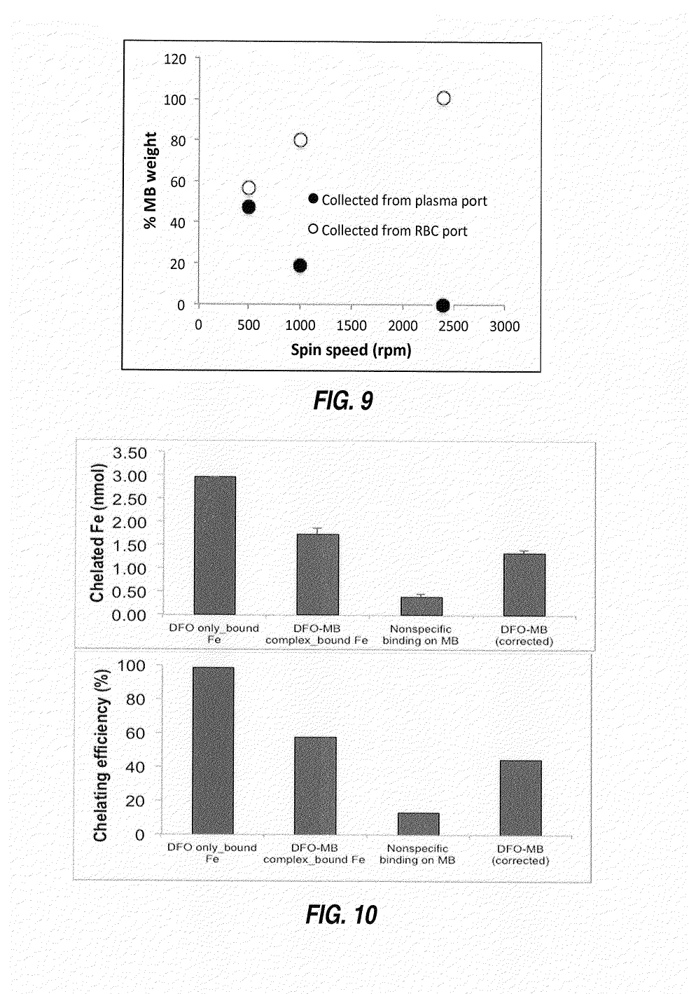

High-density Magnetic Beads (Sera-Bind Speed Beads, Thermo Scientific, Freemont, Calif.) (MB) (2 g/ml, diameter=1.3 μm) were used to demonstrate their retrieval with the Cobe Spectra Aphaerseis System. The Cobe Spectra has a blood inlet and anticoagulant inlet ports. It also has three outlet ports, which recover the blood separated in the highest, middle and lowest densities. The ports are intended for RBC, buffy coat, and plasma. The middle port was closed and pH 7.4 buffered saline (PBS) was supplied through the anticoagulant port. MB (256 mg) were washed in PBS and suspended in PBS at a final volume of 500 ml. The weight of MB was determined after collecting them magnetically in an aliquot of suspension, removing the liquid and weighing the MB. Before aphaeresis, a 25 ml aliquot of the MB/PBS solution gave a reference MB weight of 12.7 mg. The apheresis instrument was primed with PBS as usual and the flow rate of the inlet was adjusted at 32.1 ml/min and that of anticoagulant 4.6 ml/min when needed. The aphaeresis instrument was prepared to collect samples from only the plasma port (low density) and RBC port (high density). The flow rates of the two exit ports were set at 19.2 and 17.5 ml/min, respectively. The third middle density port to collect white cells and platelets was sealed. The aphaeresis was repeated three times at different speeds of centrifugation, i.e. 500, 1,000 and 2,400 rpm. Each time, 25 ml was collected from each port. The amount of MB present in the effluent collected from each of the two ports was harvested magnetically, the fluid removed and the MB weighed. The results are shown in Table 3 and the % of separation of MB from the two ports at three different spin speeds of aphaeresis is shown in FIG. 9, The results demonstrate that at the total flow rate of 36.7 ml/min, a complete separation of MB can be achieved at 2,400 rpm. Although the conditions for separation of MB and its equivalent in the plasma and blood will be different, the results strongly suggest a complete isolation of high-density nanoparticles will be feasible by adjusting the spin speed of the aphaeresis instrument and the flow rates of liquid through each port.

TABLE 3

| Spin speed | Plasma port | RBC port | Plasma port | RBC port |
|---|---|---|---|---|
| rpm | mg | mg | Wt % | Wt % |
| 2400 | 0.0 | 12.8 | 0.0 | 100.0 |
| 1000 | 2.4 | 10.2 | 19.0 | 81.0 |
| 500 | 6.0 | 7.2 | 45.5 | 54.5 |

### Example 4

### The Efficient Conjugation of Haptoglobin onto rNP and Gold Submicron Particles

Hp-rNP were formulated using 2.6 mmol 1,2-dioleoyl-sn-glycero-3-phosphocholine (DOPC), 260 μmol 1,2-dioleoyl-sn-glycero-3-phosphoethanolamine-N-(dodecanyl) (DD-DOPE), 130 μmol 1,2-dioleoyl-sn-glycero-3-phosphoethanolamine-N-[methoxy(polyethylene glycol)-1000], 20% Vol perfluoroctanylbromide (PFOB) and 80% Vol PBS. The lipids (in chloroform) were mixed, rotovap to dryness and vacuum dessicated for 3 days. They were reconstituted in 120 ml PBS. PFOB (30 ml) was added and the mixture emulsified (5000 rpm) for 1 minute to form uniform emulsion. The emulsion was homogenized at 30K×psi×10 passes to form 234 nm particles. Haptoglobin (Hp) was conjugated using EDS/S-NHS chemistry and after centrifugation the supernatant was extensively dialyzed and the free Hp determined to calculate the amount bound.

Gold nanoparticles (100 nm) were complexed with Thiol-PEG-COOH (MW 5000) overnight, then conjugated with Hp using EDC/S-NHS chemistry. The particles were pelleted at 3K×g×15 min and the supernatant collected, dialyzed and free Hp determined as above. The results for both types of particles indicate efficient Hp conjugation. Greater than 82% Hp bound to the rNP's resulting in approximately 8 nmol Hp/ml of formulation, and greater than 51% Hp bound to the gold nanoparticles, resulting in approximately 2.3 nmol Hp/ml of formulation.

### Example 5

### Iron Chelation Using Desferoxamine-Conjugated High Density Particles

Iron chelation using high density nanoparticles was studied by conjugating the iron chelator Desferoxamine (DFO) onto NHS-activated magnetic beads (MB, 2 g/ml, 1 μm diameter). The DFO and MB were reacted in 50 mM sodium borate buffer (pH 8), and the binding capacity of DFO to MB is estimated to be about 14.74 μg/mg MB. Since free iron is considered to be toxic at >60 μM, the DFO conjugated MB solution were mixed with 60 μM Fe³⁺ (Fe(NO₃)₃ 9H₂O) in PBS for 1 hour. The results show the DFO-MB complex can chelate iron, but not as well as DFO only. As shown in FIG. 10, the chelating efficiency of DFO-MB complex was about 58% compared to 99% for free DFO. The data also shows approximately 13% non-specific iron binding on DFO-free MB. Correcting for the non-specific binding results in a DFO-MB iron chelating efficiency of approximately 45%.

Although the invention has been described in detail with particular reference to the disclosed embodiments, other embodiments can achieve the same results. Variations and modifications of the present invention will be obvious to those skilled in the art and it is intended to cover all such modifications and equivalents. The entire disclosures of all patents and publications cited above are hereby incorporated by reference.

What is claimed is:

1. A method for introducing and removing high density particles from a biological fluid, the method comprising:
introducing high density particles into a biological fluid, the high density particles comprising a density greater than any naturally occurring component of the biological fluid; and
removing at least some of the high density particles from the biological fluid using aphaeresis.

2. The method of claim 1 wherein the introducing step is performed in vivo.

3. The method of claim 1 wherein the introducing step is performed extracorporeally.

4. The method of claim 3 further comprising transferring the biological fluid from a patient prior to the introducing step.

5. The method of claim 1 wherein the particles deliver oxygen in the biological fluid and scavenge carbon dioxide from the biological fluid.

6. The method of claim 1 further comprising conjugating the particles to one or more capture molecules.

7. The method of claim 6 further comprising attaching at least some of the particles to one or more targets in the biological fluid via the capture molecules prior to the removing step.

8. The method of claim 7 wherein the removing step comprises removing at least some of the attached targets from the biological fluid.

9. The method of claim 7 wherein the targets attach to an intermediary which is attached to the capture molecules.

10. The method of claim 9 wherein the intermediary comprises haptoglobin and the targets comprise hemoglobin.

11. The method of claim 7 further comprising minimizing opsonization, adherence to cells, and interaction of the particles with non-target components of the biological fluid or organs in a patient by adjusting the size and/or surface properties of the particles.

12. The method of claim 11 further comprising incorporating PEGylated and/or neutral lipids at the surface of the particles.

13. The method of claim 1 wherein the removing step is performed using a reverse-flow density gradient (RFDG) centrifuge.

14. The method of claim 13 further comprising mixing the particles with the biological fluid in a mixing chamber, the mixing chamber comprising one or more spiral tubes.

15. The method of claim 14 wherein a flow of the particles and the biological fluid is substantially lamellar within each spiral tube but not between spiral tubes.

16. The method of claim 1 further comprising automatically adjusting a removal efficiency of the particles by monitoring the concentration of particles in the biological fluid.

17. The method of claim 1 wherein each of the high density particles comprises a composition selected from the group consisting of a core comprising perfluorocarbon surrounded by a surfactant, a surface modified solid core; and an activated magnetic bead.

18. The method of claim 17 wherein the surfactant comprises a phospholipid-based monolayer.

19. The method of claim 17 wherein the solid core comprises one or more nanoparticles comprising gold, silver, titanium, iron, silica, or a ceramic.

20. The method of claim 1 further comprising returning the biological fluid to a patient after the removing step.

21. The method of claim 1 wherein the biological fluid is blood.

22. A particle capable of being removed from a biological fluid by aphaeresis, the particle comprising either a core comprising perfluorocarbon surrounded by a surfactant layer or a surface modified solid core, said particle comprising a density greater than any naturally occurring component of the biological fluid.

23. The particle of claim 22 wherein said perfluorocarbon preferably comprises perfluoroctanylbromide.

24. The particle of claim 22 wherein said surfactant layer comprises a phospholipid-based monolayer.

25. The particle of claims 24 wherein said phospholipid comprises a neutral or negatively charged headgroup.

26. The particle of claim 24 wherein said phospholipid comprises a monounsaturated phospholipid.

27. The particle of claim 24 wherein said phospholipid comprises 18:1 DOPC or 18:1 DOPA.

28. The particle of claim 22 wherein said layer comprises a co-surfactant comprising a functionalized headgroup for conjugating a capture molecule and/or a PEGylated phospholipid.

29. The particle of claim 28 wherein said PEGylated phospholipid comprises between 10 and 40 PEG subunits.

30. The particle of claim 29 wherein said. PEGylated phospholipid comprises between 14 and 25 PEG subunits.

31. The particle of claim 30 wherein said, where PEGylated phospholipid comprises PEG1000PE.

32. The particle of claim 28 wherein said co-surfactant comprises 18:1 Dodecanyl PE or 1,2-dioleoyl-sn-glycero-3-phosphoethanolamine-N-(dodecanyl).

33. The particle of claim 22 wherein said particle delivers oxygen in the biological fluid and/or scavenges carbon dioxide from the biological fluid.

34. The particle of claim 22 wherein said solid core comprises one or more nanoparticles comprising gold, silver, titanium, iron, silica, or a ceramic.

35. The particle of claim 34 wherein said solid core is surface modified with Thiol-PEG-COON or HO-PEG-N H₂ groups.

36. An apparatus for removing high density particles from a biological fluid extracorporeally, the apparatus comprising:
a mixing chamber for mixing the high density particles with the biological fluid; and
a reverse-flow density gradient (RFDG) centrifuge;
wherein a density of the high density particles is greater than any naturally occurring component of the biological fluid.

37. The apparatus of claim 36 wherein the mixing chamber comprises a spiral tube.

38. The apparatus of claim 37 comprising a plurality of spiral tubes connected in series.

39. The apparatus of claim 38 wherein a flow of the particles and the biological fluid is substantially lamellar within each spiral tube but not in a region connecting two spiral tubes.

40. The apparatus of claim 36 further comprising a pump for pumping the biological fluid through said mixing chamber and a syringe pump located before an inlet to said mixing chamber for combining said high density particles with the biological fluid.

41. The apparatus of claim 36 wherein said centrifuge comprises a variable element, said element selected from the group consisting of spin rate, number of open outlet ports, and flow rate of liquid through each outlet port.
